(19) United States
(12) Patent Application Publication
(10) Pub. No.: US 2025/0018079 A1
(43) Pub. Date: Jan. 16, 2025

(54) Systems And Methods For Air Recirculation And Volatile Composition Release

(71) Applicant: Enviroscent, Inc., Atlanta, GA (US)

(72) Inventors: Eric Mehnert, Lawrenceville, GA (US); Tamara Lyn Kullback, Atlanta, GA (US)

(21) Appl. No.: 18/707,506

(22) PCT Filed: Sep. 2, 2022
(86) PCT No.: PCT/US2022/042463

§ 371 Date: May 3, 2024

Related U.S. Application Data
(60) Provisional application No. 63/240,754, filed on Sep. 3, 2021.

(51) Int. Cl. A61L 9/12

(52) U.S. Cl. CPC A61L 9/12; A61L 2209/11; A61L 2209/134; A61L 2209/16

(57) ABSTRACT

Described is a volatile composition releasing assembly including a material including a volatile composition and a housing that has a receptacle shaped to receive the material including the volatile composition, an air intake opening, and an air output opening; the housing being shaped to create an air recirculation pattern within an interior of the housing; and wherein an airflow enters the housing through the air intake opening, and at least a portion of the airflow follows the air recirculation pattern; and wherein the portion of the airflow following the air recirculation pattern passes over an exposed surface of the material including the volatile composition at least two times before exiting the housing through the air output opening.

### CROSS REFERENCE TO RELATED APPLICATIONS

This application is related to and claims priority benefits from U.S. Provisional Application Ser. No. 63/240,754, filed Sep. 3, 2021, entitled “Enhanced Fragrance Performance by Use of Controlled Air Recirculation,” the entire contents of which are incorporated herein by reference.

### FIELD OF THE INVENTION

The field of the invention relates to systems and methods for recirculating air within a device for improved air purification and/or improved releasing of a volatile composition.

### BACKGROUND

Fragrance-releasing devices are well known and commonly used in household and commercial establishments to provide a pleasant environment for people in the immediate space. Further, aroma-driven experiences are well recognized to improve or enhance the general mood of individuals. In some instances, fragrances may trigger memories of experiences associated with the specific scent. Whether it is providing a pleasant environment, affecting a general demeanor, or triggering a nostalgic memory, a steady, long-lasting release of fragrance will ensure consumer and customer satisfaction. Candles are commonly used as fragrance-releasing devices as they can provide decoration and ambiance to a space. However, candles have a number of drawbacks including being fire hazards, risk of smoke damage or damage from the wax, lack of reusability, etc. Consumers are looking for safer, reusable alternatives to candles.

### SUMMARY

The terms “invention,” “the invention,” “this invention” and “the present invention” used in this patent are intended to refer broadly to all of the subject matter of this patent and the patent claims below. Statements containing these terms should be understood not to limit the subject matter described herein or to limit the meaning or scope of the patent claims below. Embodiments of the invention covered by this patent are defined by the claims below, not this summary. This summary is a high-level overview of various aspects of the invention and introduces some of the concepts that are further described in the Detailed Description section below. This summary is not intended to identify key or essential features of the claimed subject matter, nor is it intended to be used in isolation to determine the scope of the claimed subject matter. The subject matter should be understood by reference to appropriate portions of the entire specification of this patent, any or all drawings and each claim.

According to certain embodiments of the present invention, a volatile composition releasing assembly includes a material comprising a volatile composition and a housing comprising: a receptacle shaped to receive the material comprising the volatile composition; an air intake opening; and an air output opening; the housing being shaped to create an air recirculation pattern within an interior of the housing; and wherein an airflow enters the housing through the air intake opening, and at least a portion of the airflow follows the air recirculation pattern; and wherein the portion of the airflow following the air recirculation pattern passes over an exposed surface of the material comprising the volatile composition at least two times before exiting the housing through the air output opening.

According to certain embodiments of the present invention, an air purification assembly includes a housing comprising: an air purifying component arranged within an interior of the housing; an air intake opening; and an air output opening; the housing being shaped to create an air recirculation pattern within the interior of the housing; and wherein an airflow enters the housing through the air intake opening, and at least a portion of the airflow follows the air recirculation pattern; and wherein the portion of the airflow following the air recirculation pattern passes through or by the air purifying component at least two times before exiting the housing through the air output opening.

### BRIEF DESCRIPTION OF THE DRAWINGS

In the following detailed description, embodiments of the invention are described referring to the following figures:

FIG. 1A is a side view of a volatile composition releasing assembly, according to certain embodiments of the present invention.

FIG. 1B is a side view of a representative airflow within the volatile composition releasing assembly of FIG. 1A.

FIG. 2 is a side view of a volatile composition releasing assembly and a representative airflow within the volatile composition releasing assembly, according to certain embodiments of the present invention.

FIG. 3 is a side view of a volatile composition releasing assembly and a representative airflow within the volatile composition releasing assembly, according to certain embodiments of the present invention.

FIG. 4 is a side view of a volatile composition releasing assembly and a representative airflow within the volatile composition releasing assembly, according to certain embodiments of the present invention.

FIG. 5 is a perspective view of a volatile composition releasing assembly and a representative airflow within the volatile composition releasing assembly, according to certain embodiments of the present invention.

FIG. 6 is a perspective view of a volatile composition releasing assembly and a representative airflow within the volatile composition releasing assembly, according to certain embodiments of the present invention.

FIG. 7 is a perspective view of a volatile composition releasing assembly and a representative airflow within the volatile composition releasing assembly, according to certain embodiments of the present invention.

FIG. 8 is a side view of a volatile composition releasing assembly and a representative airflow within the volatile composition releasing assembly, according to certain embodiments of the present invention.

FIG. 9 is a side view of a multi-chamber volatile composition releasing assembly and a representative airflow within the multi-chamber volatile composition releasing assembly, according to certain embodiments of the present invention.

FIG. 10 is a side view of a multi-chamber volatile composition releasing assembly and a representative airflow within the multi-chamber volatile composition releasing assembly, according to certain embodiments of the present invention.

FIG. 11 is a side view of a multi-chamber volatile composition releasing assembly and a representative airflow within the multi-chamber volatile composition releasing assembly, according to certain embodiments of the present invention.

FIG. 12 is a perspective view of a multi-chamber volatile composition releasing assembly and a representative airflow within the multi-chamber volatile composition releasing assembly, according to certain embodiments of the present invention.

FIG. 13 is a side view of an air purifying assembly and a representative airflow within the air purifying assembly, according to certain embodiments of the present invention.

FIG. 14 is an example computing device, according to certain embodiments of the present invention.

### DETAILED DESCRIPTION

The subject matter of embodiments of the present invention is described herein with specificity to meet statutory requirements, but this description is not necessarily intended to limit the scope of the claims. The claimed subject matter may be embodied in other ways, may include different elements or steps, and may be used in conjunction with other existing or future technologies. This description should not be interpreted as implying any particular order or arrangement among or between various steps or elements except when the order of individual steps or arrangement of elements is explicitly described.

### A. Volatile Composition Releasing Assemblies

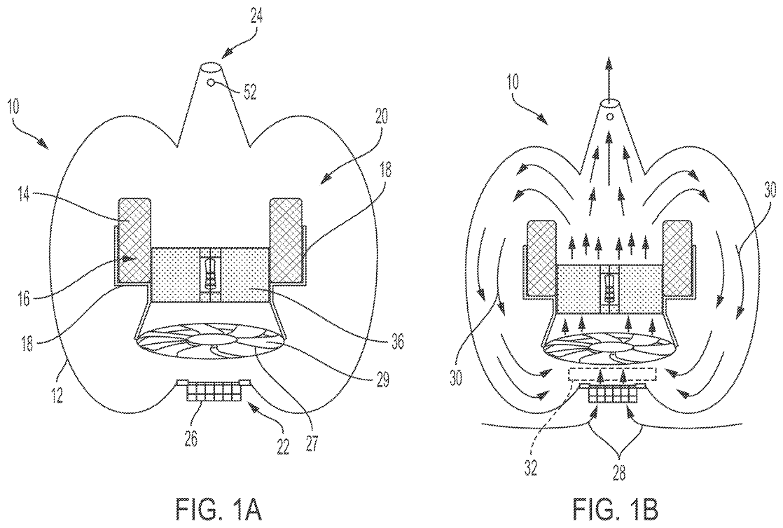

According to certain embodiments of the present invention, as shown in at least in FIGS. 1A-8, a volatile composition releasing assembly 10 includes a housing 12 and at least a material 14 comprising a volatile composition 16. The volatile composition releasing assembly 10 may be used to safely emit a strong and lasting fragrance or other by-product of the volatile composition 16 into a space with improved performance and effectiveness.

The material 14 may be formed from any material capable of absorbing, incorporating, or otherwise containing therein or thereon a scent and releasing the scent when exposed to ambient environmental conditions or active temperature and/or air applications. For example, the material 14 may include, but is not limited to, natural and/or synthetic pulp compositions; pulp compositions combined with other products, including but not limited to paper, cellulose, cellulose acetate, pulp lap, cotton linters, biological plant-derived materials (from living plants), synthesized pulp compositions, and mixed pulps; wax materials; liquid reservoirs; permeable films; gel materials; polymer material; porous material; absorptive matrices; extrudate; any suitable combination thereof, any suitable volatile component emitting material; or any other suitable absorbent material.

Further, the material 14 may be a three-dimensional object, including but is not limited to, for example, a box, a cube, a sphere, a cylinder, or any other appropriate shape. In some embodiments, the material 14 may be formed into pellets or a sheet (i.e., where the thickness is relatively small compared to the surface area) such that portions can be cut into smaller individual parts. In some embodiments, the material 14 may comprise an internal structure comprising a plurality of pores, an enclosed chamber with a permeable surface, or any other suitable configuration, which is configured to provide locations for the volatile composition 16 to be stored therein and released therefrom.

In certain embodiments, the volatile composition 16 itself forms the material 14. The volatile composition 16 may include, but is not limited to, fragrances, flavor compounds, odor-eliminating compounds, aromatherapy compounds, natural oils, water-based scents, odor neutralizing compounds, outdoor products (e.g., insect repellent), water, any suitable combination thereof, or any other suitable composition. Additional description of base materials, absorptive matrices, porosity, pulp concentrations, volatile compositions, modulating coatings or additives, additional treatments of the material and/or absorptive matrices, solvent-free fragrance dispensers, related embodiments, etc. may be found in U.S. Publication No. 2011/0262377 and U.S. Publication No. 2019/0231919, the entire contents of each of which are incorporated herein by reference.

The housing 12 of the volatile composition releasing assembly 10 may include at least one receptacle 18 arranged in an interior chamber 20 of the housing 12. The receptacle 18 may be shaped to receive the material 14 and/or hold or contain at least a portion of the material 14 within or on the receptacle 18. The receptacle 18 may cover one or more portions of the material 14 to limit the air exposure of the material 14 to only those exposed surface(s) of the material 14 not covered by the receptacle 18. In some embodiments, multiple separate materials 14 may each be received by separate, respective receptacles 18, e.g., as may be seen at least in FIGS. 1A-1B. Further, multiple separate materials 14 may each be received by a single receptacle 18. In certain embodiments, the receptacle 18 may be formed from at least one piece of solid material, at least one piece of material comprising at least one opening extending therethrough, and/or a combination of such pieces. The at least one receptacle 18 may be arranged at any suitable location within the housing 12.

In some embodiments, the housing 12 includes at least one light source 52 for emitting light from the housing 12. For example, the housing 12 may include one, two, three, four, five, six, or any suitable number of light sources 52. The at least one light source 52 may include a flickering light-emitting diode (“LED”) that may resemble the flickering of a candle flame. In certain embodiments, the housing 12 includes three light sources 52 arranged at 120 degrees from each other.

In certain embodiments, the housing 12 further comprises at least one air intake opening 22 and at least one air output opening 24, wherein one or both may extend through a surface of the housing 12 to provide openings into the interior chamber 20 of the housing. The at least one air intake opening 22 and the at least one air output opening 24 may be arranged at any suitable location on the housing 12 and at any suitable location with respect to one another. In some embodiments, as will be discussed further below with respect to at least FIGS. 9 and 11, a plurality of air intake openings 22 and/or a plurality of air output openings 24 may extend through the surface of the housing 12 to provide openings into the interior chamber 20 of the housing. In further embodiments, as will be discussed further below with respect to at least FIGS. 9-12, the housing 12 may include a plurality of interior chambers 20 and a plurality of air intake openings 22 and/or a plurality of air output openings 24, wherein one or more of the plurality of air intake openings 22 and/or one or more of the plurality of air output openings 24 extend through the surface of the housing 12 to provide openings into one or more of the plurality of interior chambers 20.

In some embodiments, the housing includes an air purifying component 26. The air purifying component 26 may be arranged in line with the air intake opening 22. For example, the air purifying component 26 may at least partially or wholly cover the air intake opening 22 and may be positioned externally, internally, or a combination thereof to the housing 12. The air purifying component 26 may include, but is not limited to, a filter, e.g., charcoal, photocatalytic, etc., electrostatic ion generation, or any other suitable air purification device or process.

The housing 12 may be formed of materials including, but not limited to, polypropylene, polycarbonate, polyethylene terephthalate, acrylic, fluorinated polyethylene, polymers, graphite composite, polyester, nylon, thermoplastic, other plastic materials, wood, metal, ceramic, stone, mineral, glass, or other suitable materials. In some embodiments, the housing 12 may be formed from or coated internally and/or externally with any suitable material that does not absorb or transmit the volatile composition 16. Such suitable materials may include but are not limited to metal, metalized films, ceramic, glass, glazed ceramics, plastic, polymers, barrier coated wood or fibers, and any other impervious material. Further, the housing 12 may be any suitable shape.

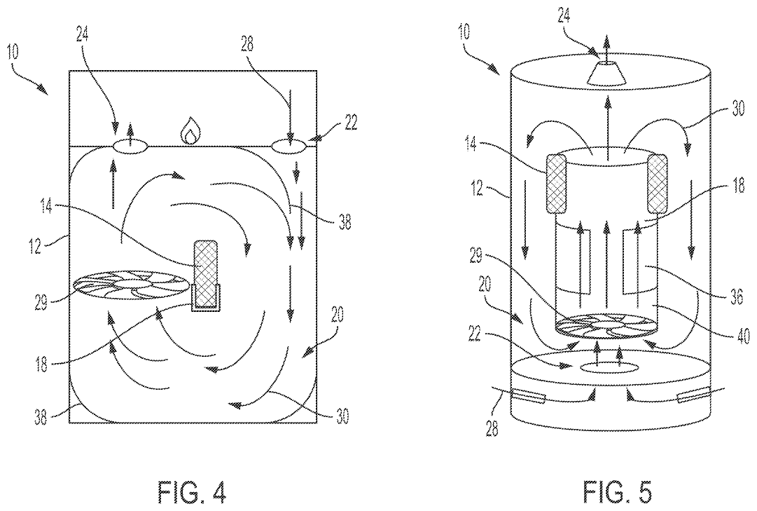

An airflow 28, as represented by the arrows in at least FIG. 1B, may enter the housing through the air intake opening 22. In some embodiments, the airflow 28 is introduced through the air intake opening 22 via at least one suction mechanism 27 that pulls the airflow 28 through the air intake opening 22. The suction mechanism 27 may be created using any suitable means, device, or method. For example, an internal air-moving device 29, such as a fan or any other suitable device, may be positioned within the housing 12 such that the air intake opening 22 is arranged on a suction side of the internal air-moving device 29 so that the internal air-moving device 29 creates a suction force that pulls the airflow 28 through the air intake opening 22. In further embodiments, the suction mechanism 27 may comprise a shape of the air intake opening 22 that creates a Venturi effect to draw the airflow 28 into the housing 12, as discussed in further detail below with respect to at least FIG. 4. In still further embodiments, the suction mechanism 27 may be positioned external to the housing 12 proximate to the air output opening 24 to pull the airflow 28 through the air output opening 24. The housing 12 may include at least one internal suction mechanism 27 positioned within the housing 12 and at least one external suction mechanism 27 positioned external to the housing 12.

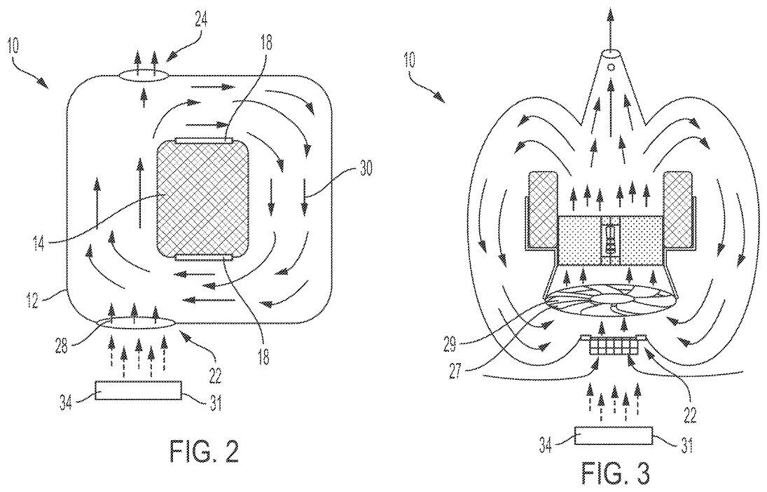

In some embodiments, as shown in at least in FIG. 2, the airflow 28 is introduced through the air intake opening 22 via at least one blowing mechanism 31 that pushes the airflow 28 into the air intake opening 22. The blowing mechanism 31 may be created using any suitable means, device, or method. For example, an external air-moving device 34 such as a car vent, an HVAC system, a fan, or any other suitable device, may be arranged outside of the housing 12 so that the external air-moving device 34 creates a blowing force that pushes airflow 28 through the air intake opening 22. In some embodiments, the external air-moving device 34 is coupled to the housing 12. In further embodiments, the external air-moving device 34 is not coupled to the housing 12 and is instead separate from the housing 12. The external air-moving device 34 may also be used to control a temperature of the airflow 28 entering and/or within the housing 12.

Additionally, as shown in at least in FIG. 3, the volatile composition releasing assembly 10 may also be positioned proximate to or coupled to the external air-moving device 34 so that the suction mechanism 27 (e.g., the internal air-moving device 29) pulls the airflow 28 through the air intake opening 22 while the blowing mechanism 31 (e.g., the external air-moving device 34) pushes the airflow 28 through the air intake opening 22.

According to certain embodiments of the present invention, as shown in at least FIGS. 1A-8, the housing 12 is shaped to create an air recirculation pattern 30 within the interior chamber 20 of the housing 12. For example, the walls of the housing 12 may be curved, angled, or otherwise shaped to create the air recirculation pattern 30. In the interior chamber 20, at least a portion of the airflow 28 that has entered through the air intake opening 22 may follow the air recirculation pattern 30. The shape of the housing 12 encourages at least the portion of the airflow 28 to move through the air recirculation pattern 30. In some embodiments, the shape of the housing 12 around the air output opening 24 may be designed to control the portion of the airflow 28 that moves towards and through the air output opening 24, which in turn controls the amount of air volume that recirculates within the housing 12 along the air recirculation pattern 30. In other words, by altering the shape of the housing 12 around the air output opening 24, a residence time of the air and, thus, the concentration of volatile composition in the airflow that exits the air output opening 24 may also be altered, as explained in more detail below. A particular housing 12 shape may be fixed for a particular application such that the alterations are performed at the manufacturing stage of the housing 12. Alternatively, the housing 12 shape may include adjustments to the shape itself, such as by adjusting aperture sizes on the openings 22, 24 or the overall volume of the housing 12 as examples, which in turn allows for more finite control of the volatile composition within a given housing 12.

As best illustrated in FIG. 1B, the portion of the airflow 28 following the air recirculation pattern 30 may move through a cycle of the air recirculation pattern 30 a plurality of times. One cycle of the air recirculation pattern 30 begins at a starting point, e.g., block 32 (though the starting point could be any point along the air recirculation pattern 30) within the interior chamber 20 and moves along a path within the housing 12. One cycle of the air recirculation pattern 30 is complete when the portion of the airflow 28 has moved from the starting point, around the entire path of the air recirculation pattern 30, and has returned to the starting point. The portion of the airflow 28 may move through at least one cycle of the air recirculation pattern 30 before exiting the interior chamber 20 through the air output opening 24. In some embodiments, the portion of the airflow 28 may move through a plurality of cycles of the air recirculation pattern 30 before exiting the interior chamber 20 through air output opening 24.

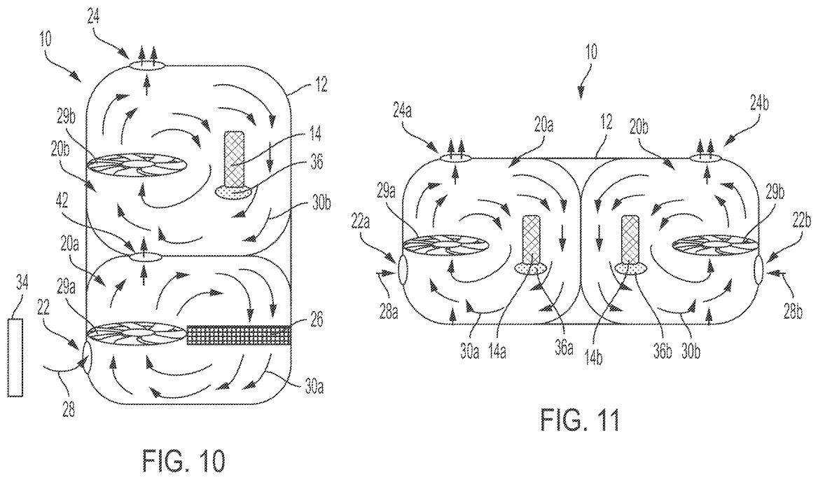

In certain embodiments, there may be multiple separate air recirculation patterns 30 within the interior chamber 20 of the housing 12. For example, a first air recirculation pattern 30 may be formed on a first side of the interior chamber 20 and a second air recirculation pattern 30 may be formed on a second side of the interior chamber 20. A first portion of the airflow 28 may move through the first air recirculation pattern 30 and a second portion of the airflow 28 may move through the second air recirculation pattern 30. In some embodiments, as best illustrated in FIG. 9, portions of the first air recirculation pattern 30 and the second air recirculation pattern 30 may overlap, e.g., in a center portion of the interior chamber 20. In further embodiments, as best illustrated in FIGS. 10-12, the portion of the airflow 28 may move through more than one air recirculation pattern 30. For example, as best illustrated in FIG. 10, the first portion of the airflow 28 may move through the first air recirculation pattern 30 for one cycle and some or all of the first portion of the airflow 28 may move through the second air recirculation pattern 30 for a second cycle thereafter (series arrangement). In a further example, as best illustrated in FIG. 11, a first portion of the airflow 28a may move through the first air recirculation pattern 30 for at least one cycle, while a second portion of the airflow 28b may simultaneously move through the second air recirculation pattern 30b for at least one cycle (parallel arrangement).

As the portion of the airflow 28 moves within the housing 12 following the air recirculation pattern 30, the portion of the airflow 28 passes over an exposed surface of the material 14 so that molecules of the volatile composition 16 evaporate from the material 14 and move with the portion of the airflow 28 through the air recirculation pattern 30. In some embodiments, the portion of the airflow 28 may pass over a plurality of exposed surfaces of the material 14 during a single cycle of the air recirculation pattern 30. In further embodiments, the portion of the airflow 28 may pass over at least one exposed surface of the material 14 during a single cycle of the air recirculation pattern 30.

Increasing the number of times the portion of the airflow 28 passes over an exposed surface of the material 14 before the portion of the airflow 28 exits the interior chamber 20 and the housing 12 through the air output opening 24 increases the concentration of molecules of the volatile composition 16 that move with the portion of the airflow 28 and exit through the air output opening 24. Therefore, the portion of the airflow 28 exiting the housing 12 contains an increased amount of molecules of the volatile composition 16 than if the portion of the airflow 28 had only passed over one exposed surface of the material 14 one time. In certain embodiments, the amount of molecules of the volatile composition 16 present in the portion of the airflow 28 exiting the housing 12 may increase with each pass over the exposed surface(s) of the material 14 up to a point of saturation. Increasing the amount of molecules of the volatile composition 16 in the portion of the airflow 28 exiting through the air output opening 24 will increase the strength and potency of the fragrance or other by-product of the volatile composition 16 emitted from the volatile composition releasing assembly 10.

In certain embodiments, the amount of molecules evaporating from the volatile composition 16 into the portion of the airflow 28 may depend on an air impingement angle that the portion of the airflow 28 makes with the exposed surface of the material 14. For example, the portion of the airflow 28 just grazing the exposed surface of the material 14 and thus having an air impingement angle that is almost parallel with the exposed surface may have a different release rate of molecules from the volatile composition 16 versus the portion of the airflow 28 hitting the exposed surface at a higher angle. In addition, the air impingement angle may also affect the speed of the air recirculation and the number of recirculation cycles before the portion of the airflow 28 exit the housing 12.

In some embodiments, the housing 12 includes at least one energy source 36. The energy source 36 may be a heating element, a cooling element, or a chemical element, e.g., an element that creates an exothermic reaction. The at least one energy source 36 may be arranged at any suitable location within the housing 12. For example, the at least one energy source 36 may at least partially cover the air intake opening 22, may be arranged in line with at least one of the air intake opening 22, the internal air-moving device 29, the material 14, etc., or may be arranged along the walls of the housing 12.

The at least one energy source 36 may be used to control a temperature of the air, e.g., a temperature of at least the portion of the airflow 28, within the housing. For example, the heating element may be a resistor type heater arranged in a center of fins formed from a metal or other conductive material that radiate outwards from the resistor. As the airflow 28, or in some instances at least the portion of the airflow 28, travels through the resistor type heater, the airflow 28 picks up the heat from the fins to increase the temperature of the airflow 28. The at least one energy source 36 is shaped and arranged within the housing 12 so that the at least one energy source 36 does not restrict the movement of the airflow 28 within the housing 12.

In some embodiments, the airflow 28 passes through, across, or proximate to a surface of the at least one energy source 36, and the temperature of the airflow 28 changes based on the temperature of the at least one energy source 36. Additionally, the at least one energy source 36 may be positioned in the path of the air recirculation pattern 30 so that the portion of the airflow 28 interacts with the at least one energy source 36 at least once during one cycle of the air recirculation pattern 30. As the portion of the airflow 28 moves through multiple cycles of the air recirculation pattern 30, the portion of the airflow 28 may interact with the at least one energy source 36 each time until the portion of the airflow 28 reaches an equilibrium temperature. In certain embodiments, the equilibrium temperature is controlled by the temperature of the at least one energy source 36 and the ratio of the portion of the airflow 28 moving through the air recirculation pattern 30 versus the portion exiting the housing 12 through the air output opening 24.

In some embodiments, the at least one energy source 36, or in some examples a separate, additional energy source 36, may be used to control a temperature of the material 14. For example, the energy source 36 may be arranged integrally within or coupled to the material 14 itself, the receptacle 18, or a combination thereof. The receptacle 18 may be formed of conductive materials to facilitate the heat transfer from the energy source 36 to the material 14 or may be formed of insulative materials to limit the heat transfer from the energy source 36 to the material 14. In certain embodiments, the at least one energy source 36 may be used only to heat or cool the airflow 28 to control only the temperature of the airflow 28, may be used only to heat or cool the material 14 to control only the temperature of the material 14, or may be used to heat or cool both the airflow 28 and the material 14 to control the temperature of both. For example, the airflow 28 may not be heated while the material 14 is heated by the at least one energy source 36.

Additionally, the at least one energy source 36 may be arranged external to the housing 12. In some embodiments, the external air-moving device 34 may function as the at least one energy source 36. As discussed above, the external air-moving device 34 may also be used to control the temperature of the airflow 28 entering and/or within the housing 12. Further, the at least one energy source 36 may be in direct or indirect contact with an external surface of the housing 12 to control a temperature of the housing 12 and thus the temperature of the airflow 28 and/or material 14 within the housing 12. For example, the at least one energy source 36 may be a clothes dryer, a refrigerator, an HVAC system, etc. to which the volatile composition releasing assembly 10 may be coupled, e.g., via a magnet, a releasable clip, snap-fit, hook & loop, or any other suitable repeatedly-releasable or non-repeatedly-releasable connector, to control a temperature of the housing 12.

In certain embodiments, the volatile composition releasing assembly 10 and any of its components may be powered by a power source. Examples of power sources include power plants, solar panels, wind turbine generators, hydropower generators, fuel cells, batteries, rechargeable batteries, AC/DC power connectors, or any other suitable power source. The components, e.g., the air purifying component 26, the suction mechanism 27, the internal air-moving device 29, the blowing mechanism 31, the external air-moving device 34, the energy source 36, and/or the light source 52, etc. may be powered by the same power source or different power sources if the component requires power.

In some embodiments, such as the embodiments illustrated in FIG. 4, the air intake opening 22 and the air output opening 24 are positioned along the same side of the housing 12. The receptacle 18 and the material 14 may be arranged at a center of the housing 12. The internal air-moving device 29 may be arranged within the interior chamber 20 of the housing 12 and/or the external air-moving device 34 may be arranged outside of the housing 12 so that the suction mechanism 27 and/or the blowing mechanism 31 contribute to moving the portion of the airflow 28 through one or a plurality of cycles of the air recirculation pattern 30.

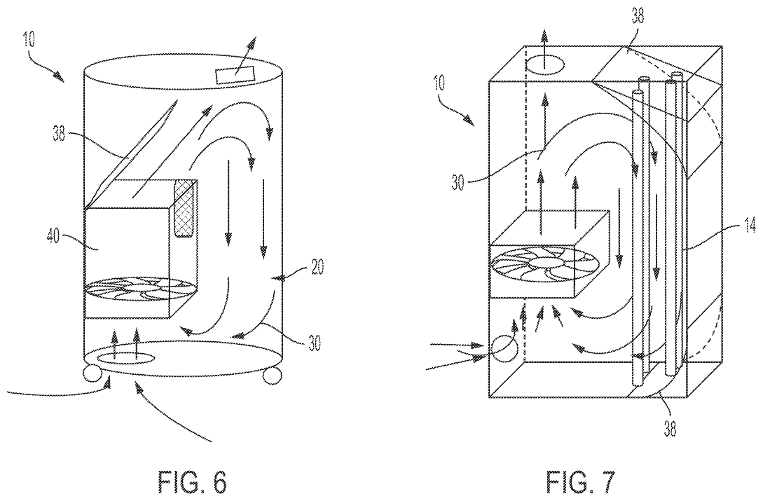

In certain embodiments, such as the embodiments illustrated in FIGS. 6-7, the housing 12 may include one or more air directing members 38 arranged within the interior chamber 20. The air directing members 38 may be used to assist in creating the air recirculation pattern 30 based on the positioning of the air directing member 38 within the housing 12. Further, at least one of the air directing members 38 may be located to create a passageway in line with the air intake opening 22 so as to create the suction force via the Venturi effect to pull the airflow 28 through the air intake opening 22 and into the interior chamber 20.

In some embodiments, such as the embodiments illustrated in FIG. 5, a passageway 40 is formed within the interior chamber 20 and arranged at a central location within the interior chamber 20. At least a portion of the passageway 40 may form the receptacle 18 for receiving the material 14. The internal air-moving device 29 may be positioned proximate to, at the inlet and/or exit of, or within the passageway 40. Additionally, the at least one energy source 36 may be integrally formed with or coupled to at least a portion of the passageway 40.

In some embodiments, such as the embodiments illustrated in FIG. 6, the passageway 40 is arranged along a side of the interior chamber 20 and an air directing member 38 is integrally formed with, coupled to, or positioned proximate to the passageway 40 to assist in creating the air recirculation pattern 30.

In some embodiments, such as the embodiments illustrated in FIG. 7, the at least one air directing member 38 forms the receptacle for receiving at least a portion of the material 14 while also assisting in creating the air recirculation pattern 30.

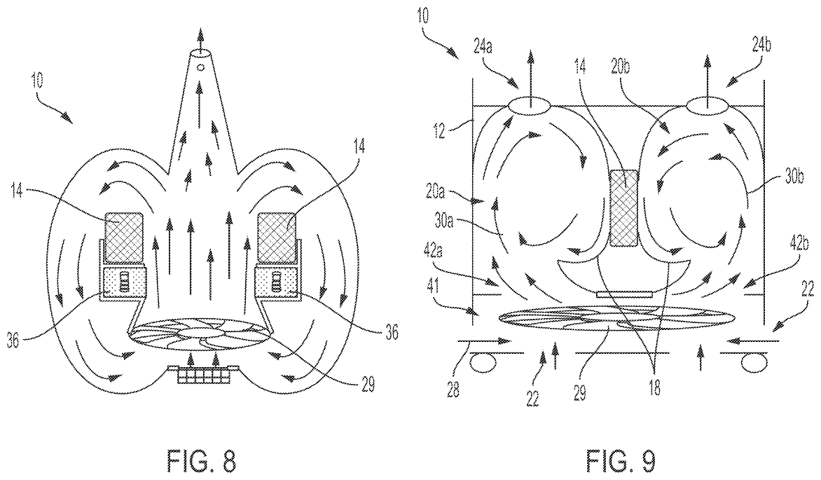

In some embodiments, such as the embodiments illustrated in FIG. 8, the at least one energy source 36 is arranged out of the path of the airflow 28, e.g., the at least one energy source 36 is not positioned in line with the internal air-moving device 29.

### B. Adjustability of Volatile Composition Releasing Assemblies

According to certain embodiments, such as the embodiments illustrated in FIGS. 1A-8, various features of the volatile composition releasing assembly 10 may be adjustable to control the concentration of the molecules of the volatile composition 16 in the portion of the airflow 28 as well as the temperature of the portion of the airflow 28.

In some embodiments, the material 14 and arrangement of the material 14 within the housing 12 is adjustable. For example, the amount of material 14, the ratio of volume to surface area of the material 14, the formulation of the material 14 and/or the volatile composition 16, the placement of the material 14 proximate to the internal air-moving device 29 and/or the at least one energy source 36, the amount of exposed surface(s) of material 14, the air impingement angle on the exposed surface(s) of material 14, coatings and/or other permeable surface of material 14, etc. may each be adjusted, either individually or in combination, to create an optimal performance of the volatile composition releasing assembly 10. In certain embodiments, the volatile composition 16 may be a scented composition, and such adjustments may adjust the load and fragrance release of the scented composition from the material 14.

Further, the arrangement of various elements within the housing 12, e.g., the receptacle 18, the internal air-moving device 29, the at least one energy source 36, the air directing member 38, and/or the passageway 40, etc. are adjustable. Adjusting these elements may affect the concentration of the molecules of the volatile composition 16 in the portion of the airflow 28, the temperature of the portion of the airflow 28, and/or the temperature of the material 14. For example, a plurality of receptacles 18 may be arranged at various positions in relation to the at least one energy source 36. This arrangement may affect the amount of heat contact the material 14 has with the at least one energy source 36, thus affecting the temperature of the material 14 as well as the exposure of the material 14 to the airflow 28. Controlling the temperature of the material 14 and the exposure of the material 14 to the airflow 28 may control the evaporation and thus concentration of the molecules of the volatile composition 16 in the portion of the airflow 28.

Where the at least one energy source 36 heats the portion of the airflow 28 with each cycle of the recirculation, the temperature of the portion of the airflow 28 will continue to increase until the heat loss from the air output opening 24 equals the heat gain from the at least one energy source 36. This temperature may control how much fragrance or by-product of the volatile composition 16 is released into the portion of the airflow 28 inside the interior chamber(s) 20. As the temperature increases, the amount of fragrance given off into the recirculation chamber is increased. The concentration of fragrance continues to increase until the amount of fragrance given off by the material 14 equals the amount lost through the air output opening 24. The concentration of the fragrance being lost at the air output opening 24 will be at a higher level with warmer air than it will be with ambient or cooler air.

The converse is true for cooling. Specifically, where the at least one energy source 36 cools the portion of the airflow 28 with each cycle of the recirculation, the temperature of the portion of the airflow 28 will continue to drop until the heat added by the additional airflow 28 through the air input opening 22 equals the heat removed from the at least one energy source 36. As the temperature decreases, the amount of fragrance given off into the recirculation chamber is decreased. The concentration of fragrance continues to decrease until the amount of fragrance given off by the material 14 equals the amount lost through the air output opening 24. The concentration of the fragrance being lost at the air output opening 24 will be at a lower level with cooler air than it will be with ambient or warmer air. Increasing the air intake and/or exhaust will affect this dynamic and a new equilibrium will be reached.

In some embodiments, adjustments made to at least one of the air intake opening 22, the air output opening 24, the internal air-moving device 29, the external air-moving device 34, or the shape of the housing 12 itself may affect the amount of airflow moving into and out of the housing 12 and the amount of air recirculation that occurs within the housing 12. Further, the relationship between the air intake opening 22, the air output opening 24, the internal air-moving device 29, and/or the external air-moving device 34 may also affect the amount of air recirculation that occurs within the housing 12.

At least one of the air intake opening 22 or the air output opening 24 may be adjustable to control the amount of the airflow 28 entering and exiting the housing 12. In some embodiments, the air intake opening 22 and the air output opening 24 are each adjustable by changing at least one of the size of the air intake opening 22 and/or the air output opening 24, the shape of the air intake opening 22 and/or the air output opening 24, the shape or height of any passageway leading into the interior chamber 20 from the air intake opening 22 and/or out of the interior chamber 20 to the air output opening 24.

For example, decreasing the size of the air output opening 24 will result in less airflow exiting the housing 12. Thus, the portion of the airflow 28 moving through the recirculation pattern 30 will remain within the housing 12 for a longer time period. In some embodiments and as a non-limiting example, the volume of the interior chamber 20 may be 0.5 cubic feet and the housing 12 may include the internal air-moving device 29 that is circulating the airflow at 5.0 cubic feet per minute and the air output opening 24 is sized to permit 1.0 cubic foot per minute of the airflow 28 to exit the housing 12. Therefore, in the non-limiting example, the average resonance time of the airflow 28 within the housing 12 is 30 seconds and the airflow 28 will move through an average of 2.5 cycles of the recirculation pattern 30 before exiting the housing 12. In the non-limiting example, if the size of the air output opening 24 is decreased to permit 0.5 cubic foot per minute of the airflow 28 to exit the housing 12, then the average resonance time of the airflow 28 within the housing 12 is 1 minute, and the airflow 28 will move through an average of 5 cycles of the recirculation pattern 30 before exiting the housing 12. In the non-limiting example, if the size of the air output opening 24 is decreased to permit 0.1 cubic foot per minute of the airflow 28 to exit the housing 12, then the average resonance time of the airflow 28 within the housing 12 is 5 minutes and the airflow 28 will move through an average of 25 cycles of the recirculation pattern 30 before exiting the housing 12.

By remaining within the housing 12 for longer time, the portion of the airflow 28 may move through an increased number of cycles of the recirculation pattern 30 and the concentration of the molecules of the volatile composition 16 in the portion of the airflow 28 that exits the housing 12 may be increased. The concentration of the molecules of the volatile composition 16 in the portion of the airflow 28 may increase because increasing the number of cycles of the recirculation pattern 30 may also increase the number of times the portion of the airflow 28 passes over the exposed surface of the material 14 and/or because increasing the number of cycles of the recirculation pattern 30 may also increase or decrease the temperature of the portion of the airflow 28, which may allow for more concentration of the molecules of the volatile composition 16 within the portion of the airflow 28. Increasing size of the air output opening 24 will result in more airflow exiting the housing 12 and decrease the amount of recirculation.

In some embodiments, the shape or height of the passageway leading out of the interior chamber 20 through the air output opening 24 may affect the amount of time the portion of the airflow 28 remains within the housing 12. For example, increasing the height of the passageway or narrowing the shape of the passageway may affect the velocity of the portion of the airflow 28 traveling through the passageway to the air output opening 24. Additionally, the shape of the interior chamber 20 and/or the overall shape of the housing 12 may be adjustable, as discussed previously either at the manufacturing stage or at the consumption stage by the user. The shape of the interior chamber 20 and/or the overall shape of the housing 12 may control how much recirculation occurs within the volatile composition releasing assembly 10 to control the amount of fragrance or other by-product of the volatile composition 16 that is emitted from the housing 12.

In certain embodiments, the size and/or speed of the internal air-moving device 29 and/or the external air-moving device 34 may affect the amount of time the portion of the airflow 28 remains within housing 12. For example, increasing the size and/or speed of the internal air-moving device 29 and/or the external air-moving device 34, and thereby increasing an internal air speed of the portion of the airflow 28, may cause the portion of the airflow 28 to move through an increased number of cycles of the recirculation pattern 30 during the same amount of time. Thus, the temperature and/or the concentration of the molecules of the volatile composition 16 in the portion of the airflow 28 may increase. In some embodiments, the concentration may be further adjusted by increasing or decreasing the size of the air opening 24 in combination with decreasing or increasing the size and/or speed of the internal air-moving device 29 and/or the external air-moving device 34 and/or with increasing or decreasing a temperature of the portion of the airflow 28 using the energy source 36.

The embodiments illustrated with respect to FIGS. 1A-8 may be combined with any, some, or all of the embodiments described within this application.

### C. Multi-Chamber Volatile Composition Releasing Assemblies

According to certain embodiments of the present invention, such as the embodiments illustrated in FIGS. 9-12, may also include multiple air recirculation chambers.

In some embodiments, such as the embodiments illustrated in FIG. 9, the housing 12 may include multiple, separate interior chambers 20. In some embodiments, the at least one air intake opening 22 is arranged to provide access to an intake interior chamber 41. Additionally, the internal air-moving device 29 may be positioned at least partially within the intake interior chamber 41. The suction force created by the internal air-moving device 29 pulls an airflow 28 through the at least one air intake opening 22 and into the intake interior chamber 41. A plurality of intermediate openings 42, e.g., a first intermediate opening 42a and a second intermediate opening 42b, may each lead from the intake interior chamber 41 to additional, separate interior chambers, e.g., a first interior chamber 20a and a second interior chamber 20b, so that a portion of the airflow 28 may move through each of the plurality of intermediate openings 42 and into respective first and second interior chambers 20a and 20b. While three interior chambers 20a, 20b, and 41 are shown in FIG. 9, the housing 12 may include two, four, five six, seven, or any other suitable number of interior chambers.

In some embodiments, the walls of the first and second interior chambers 20a and 20b may form the receptacle 18 for receiving the material 14. The material 14 may be arranged so that there is an exposed surface of the material 14 in both the first and second interior chambers 20a and 20b. In further embodiments, each of the first and second interior chambers 20a and 20b may include a receptacle 18 and material 14.

The shape of the first and second interior chambers 20a and 20b may be designed to create a first air recirculation pattern 30a within the first interior chamber 20a and a second air recirculation pattern 30b within the second interior chamber 20b. The first and second interior chambers 20a and 20b may each include a respective air output opening 24, e.g., a first air output opening 24a and a second air output opening 24b.

In some embodiments, such as the embodiments illustrated in FIG. 10, the housing 12 may include a first interior chamber 20a and a second interior chamber 20b, wherein the first interior chamber 20a and the second interior chamber 20b are arranged in series with one another. For example, the airflow 28 may enter the first interior chamber 20a through the air intake opening 22. At least a portion of the airflow 28 may recirculate through any suitable number of cycles within the first interior chamber 20a following the first air recirculation pattern 30a. After traveling through at least two cycles of the first air recirculation pattern 30a, the portion of the airflow 28 may proceed through the intermediate opening 42. After moving through the intermediate opening 42, at least the portion of the airflow 28 may enter the second interior chamber 20b. At least a portion of the airflow 28 may recirculate through any suitable number of cycles within the second interior chamber 20b following the second air recirculation pattern 30b. After traveling through at least two cycles of the second air recirculation pattern 30b, the portion of the airflow 28 may exit the housing 12 through the air output opening 24.

In some embodiments, each interior chamber, e.g., first interior chamber 20a and second interior chamber 20b, may have a different function. For example, the first interior chamber 20a may include at least one air purifying component 26 to serve the function of purifying the airflow 28. As at least the portion of the airflow 28 moves through the first air recirculation pattern 30a, the portion of the airflow 28 will move through or by the at least one air purifying component 26. With each cycle that the portion of the airflow 28 moves through, the air purifying component 26 may provide additional cleaning to the portion of the airflow 28. Further, the first interior chamber 20a may include at least one first internal air-moving device 29a that may contribute to pulling the airflow 28 through the air intake opening 22 and moving at least the portion of the airflow 28 through the first air recirculation pattern 30a. In certain embodiments, the at least one first internal air-moving device 29a and/or an external air-moving device 34 may be used to push or pull the airflow 28 through the air intake opening 22.

The second interior chamber 20b may include at least one material 14 to serve the function of saturating the airflow 28 with molecules of the volatile composition 16. As at least a portion of the airflow 28 moves through the second air recirculation pattern 30b, the portion of the airflow 28 will pass by at least one exposed surface of the material 14. With each cycle that the portion of the airflow 28 moves through, the portion of the airflow 28 may become increasingly saturated with molecules of the volatile composition 16. Further, the second interior chamber 20b may include at least one second internal air-moving device 29b that may contribute to pulling at least a portion of the airflow 28 through the intermediate opening 42 and moving at least a portion of the airflow 28 through the second air recirculation pattern 30b. Additionally, at least one of the first interior chamber 20a or the second interior chamber 20b may include at least one energy source 36. In some embodiments, the at least one energy source 36 also forms the receptacle 18 for receiving the at least one material 14.

In some embodiments, such as the embodiments illustrated in FIG. 11, the housing 12 may include a first interior chamber 20a and a second interior chamber 20b where the first interior chamber 20a and the second interior chamber 20b are arranged in parallel to one another. For example, the first interior chamber 20a may include a first material 14a, a first air intake opening 22a, a first air output opening 24a, a first airflow 28a, a first internal air-moving device 29a, a first air recirculation pattern 30a, and a first energy source 36a. The second interior chamber 20b may include a second material 14b, a second air intake opening 22b, a second air output opening 24b, a second airflow 28b, a second internal air-moving device 29b, a second air recirculation pattern 30b, and a second energy source 36b. In some embodiments, the elements included in the first interior chamber 20a differ from the elements included in the second interior chamber 20b. In further embodiments, a first air purifying component and a second air purifying component may be incorporated into the volatile composition releasing assembly 10 proximate the respective first air intake opening 22a and the second air intake opening 22b.

The first airflow 28a may enter the first interior chamber 20a through the first air intake opening 22a. A portion of the first airflow 28a will move along the first air recirculation pattern 30a for at least two cycles before exiting the first interior chamber 20a through the first air output opening 22a. The second airflow 28b may enter the second interior chamber 20b through the second air intake opening 22b. A portion of the second airflow 28b will move along the second air recirculation pattern 30b for at least two cycles before exiting the second interior chamber 20b through the second air output opening 24b. In some embodiments, the first interior chamber 20a and the first air recirculation pattern 30a is completely separate from the second interior chamber 20b and the second air recirculation pattern 30b.

In certain embodiments, the first volatile composition 16a of the first material 14a is a first fragrance and the second volatile composition 16b of the second material 14b is a second fragrance, where the first fragrance is different from the second fragrance. The volatile composition releasing assembly 10 may be controlled either manually or using a processor so that only one of the first fragrance or the second fragrance is emitted at a time from the volatile composition releasing assembly 10. In further embodiments, the volatile composition releasing assembly 10 may be controlled either manually or using the processor so that the first fragrance and the second fragrance are emitted at the same time.

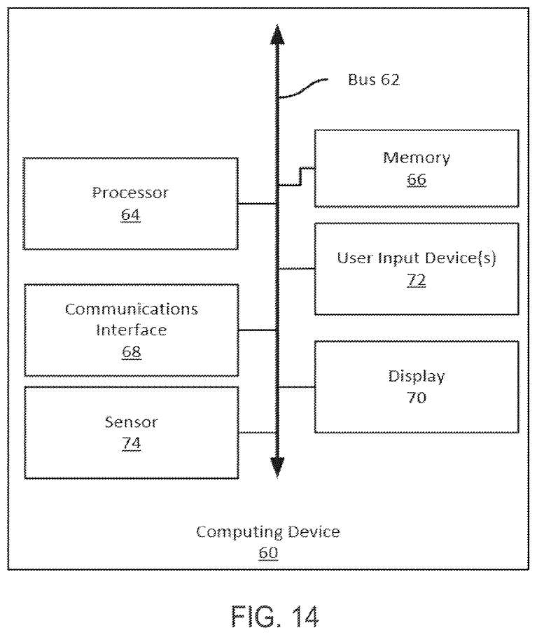

In some embodiments, such as the embodiments illustrated in FIG. 14, the volatile composition releasing assembly 10 may include a computing device 60 suitable for use in example assemblies according to this disclosure. The example computing device 60 includes a processor 64 which is in communication with the memory 66 and other components of the computing device 60 using one or more communications buses 62. The processor 64 executes processor-executable instructions stored in the memory 66 to assist with controlling the volatile composition releasing assembly 10 and any of the components within the volatile composition releasing assembly 10. The computing device 60 may also include one or more user input devices 72, such as a keyboard, mouse, touchscreen, microphone, etc., to accept user input. The computing device 60 may also include a display 70 to provide visual output to a user.

The computing device 60 also includes a communications interface 68. In some examples, the communications interface 68 may enable communications using one or more networks, including a local area network (“LAN”); wide area network (“WAN”), such as the Internet; metropolitan area network (“MAN”); point-to-point or peer-to-peer connection; etc. Communication with other devices may be accomplished using any suitable networking protocol. For example, one suitable networking protocol may include the Internet Protocol (“IP”), Transmission Control Protocol (“TCP”), User Datagram Protocol (“UDP”), or combinations thereof, such as TCP/IP or UDP/IP.

While some examples of methods and devices herein are described in terms of software executing on various machines, the methods and devices may also be implemented as specifically-configured hardware, such as field-programmable gate array (FPGA) specifically to execute the various methods. For example, examples can be implemented in digital electronic circuitry, or in computer hardware, firmware, software, or in a combination thereof. In one example, a device may include a processor or processors. The processor includes a computer-readable medium, such as a random access memory (RAM) coupled to the processor. The processor executes computer-executable program instructions stored in memory, such as executing one or more computer programs. Such processors may include a microprocessor, a digital signal processor (DSP), an application-specific integrated circuit (ASIC), field programmable gate arrays (FPGAs), and state machines. Such processors may further include programmable electronic devices such as PLCs, programmable interrupt controllers (PICs), programmable logic devices (PLDs), programmable read-only memories (PROMs), electronically programmable read-only memories (EPROMs or EEPROMs), or other similar devices.

Such processors may include, or may be in communication with, media, for example computer-readable storage media, that may store instructions that, when executed by the processor, can cause the processor to perform the steps described herein as carried out, or assisted, by a processor. Examples of computer-readable media may include, but are not limited to, an electronic, optical, magnetic, or other storage device capable of providing a processor, such as the processor in a web server, with computer-readable instructions. Other examples of media include, but are not limited to, a floppy disk, CD-ROM, magnetic disk, memory chip, ROM, RAM, ASIC, configured processor, all optical media, all magnetic tape or other magnetic media, or any other medium from which a computer processor can read. The processor, and the processing, described may be in one or more structures, and may be dispersed through one or more structures. The processor may include code for carrying out one or more of the methods (or parts of methods) described herein.

In some examples, the processor 64 is part of a computerized control system for the volatile composition releasing assembly 10 that controls the adjustment of the components of the volatile composition releasing assembly 10 as described above to adjust the speed and/or volume of the airflow 28; the temperature of the housing 12, the airflow 28, and/or the material 14; the size and/or shape of the openings 22, 24; etc. The computerized control system may enable a plurality of volatile composition releasing assemblies 10 to be networked together and be controllable using the same computerized control system. Further, the computerized control system may enable control of one or more volatile composition releasing assemblies 10 using programming, smart technology, and/or computer or mobile apps. For example, a program implemented by the processor 64 may activate the volatile composition releasing assembly 10 at different times and/or on different days with different settings.

In some examples, the processor 64 is part of a computerized control system for the volatile composition releasing assembly 10 that controls the adjustment of the components of the volatile composition releasing assembly 10 as described above to adjust the speed and/or volume of the airflow 28; the temperature of the housing 12, the airflow 28, and/or the material 14; the size and/or shape of the openings 22, 24; etc. based on information relating to the material 14 and/or the volatile composition 16. The volatile composition releasing assembly 10 may include a measuring device that measures characteristics of the material 14 and/or the volatile composition 16, e.g., weight, volume, etc., over time and sends data containing the measured characteristics to processor 64. Based on this measurement data, the processor 64 may control the adjustment of the components of the volatile composition releasing assembly 10 in real time as the measured characteristics change. For example, if the volatile composition 16 starts to evaporate or release from the material 14 less later in the life of the material 14, the processor 64 may adjust the speed and/or volume of the airflow 28; the temperature of the housing 12, the airflow 28, and/or the material 14; the size and/or shape of the openings 22, 24; etc. based on the measurement data received to provide an improved release of the volatile composition 16.

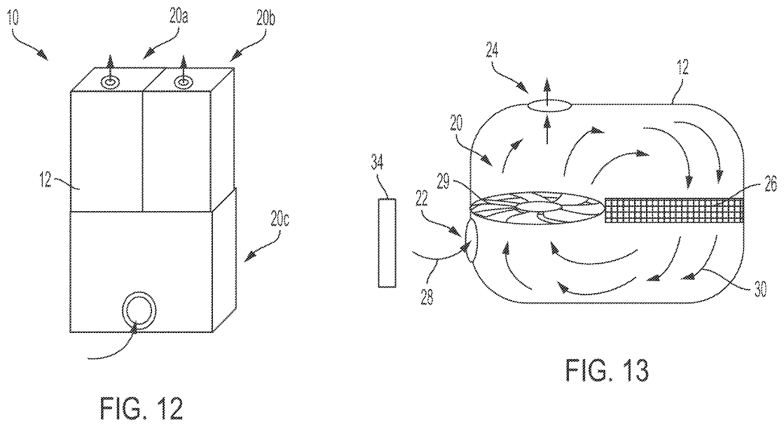

In some embodiments, such as the embodiments illustrated in FIG. 12, the volatile composition releasing assembly 10 may be the same that described in reference to FIG. 11 with respect the first interior chamber 20a and the second interior chamber 20b, which are arranged parallel to one another. The housing 12 also includes a third interior chamber 20c that functions to purify the airflow before it enters the respective first interior chamber 20a and the second interior chamber 20b. The third interior chamber 20c may include a third airflow, a third internal air-moving device, a third air recirculation pattern, and a third air purifying component. In certain embodiments, there may be any number or combination of air purifying interior chamber(s), volatile composition interior chamber(s), or air purifying and volatile composition interior chamber(s).

The embodiments illustrated with respect to FIGS. 9-12 may be combined with any, some, or all of the embodiments described within this application.

### D. Air Purification Assemblies

According to certain embodiments of the present invention, the material 14 and volatile composition 16 is excluded from the housing 12 to form an air purification assembly 50. In these embodiments, as illustrated in FIG. 13, the airflow 28 may enter the interior chamber 20 through the air intake opening 22. At least a portion of the airflow 28 may recirculate through any suitable number of cycles within the interior chamber 20 following the air recirculation pattern 30 and passing through or by the air purifying component 26 at least once during each cycle. After traveling through at least two cycles of the air recirculation pattern 30, the portion of the airflow 28 may proceed through the air output opening 24 to exit the housing 12. The recirculation of the portion of the airflow 28 within the air purification assembly 50 may provide increase air purification as compared to a device where the airflow only passes through or by an air purifying component just once before exiting the device. In some embodiments, the air purification assembly 50 is a stand-alone device.

In some embodiments, the air purification assembly 50 may include a plurality of interior chambers 20. For example, a first interior chamber of the air purification assembly 50 may include an air filter through which the portion of the airflow 28 may recirculate to purify the portion of the airflow 28. The portion of the airflow 28 may then flow into a second interior chamber of the air purification assembly 50 that may include photocatalytic oxidation (“PCO”), which may further purify the portion of the airflow 28 recirculating within the second interior chamber before the portion of the airflow 28 exits the housing 12. The multi-chamber air purification assembly 50 may have the same or similar structural arrangement to the volatile composition releasing assembly 10 shown in at least in FIGS. 9-12.

The embodiments illustrated with respect to FIG. 13 may be combined with any, some, or all of the embodiments described within this application. For example, the material 14 and the volatile composition 16 may both be included within the same interior chamber 20 of the housing 12 of the air purification assembly 50 so that the portion of the airflow 28 following the air recirculation pattern 30 passes through or by the air purifying component 26 and passes over an exposed surface of the material 14 at least once during each cycle.

In the following, further examples are described to facilitate understanding of aspects of the invention:

Example 1 is a volatile composition releasing assembly (which may incorporate features of any of the subsequent examples) comprising: a material comprising a volatile composition; and a housing comprising: a receptacle shaped to receive the material comprising the volatile composition; an air intake opening; and an air output opening; the housing being shaped to create an air recirculation pattern within an interior of the housing; and wherein an airflow enters the housing through the air intake opening, and at least a portion of the airflow follows the air recirculation pattern; and wherein the portion of the airflow following the air recirculation pattern passes over an exposed surface of the material comprising the volatile composition at least two times before exiting the housing through the air output opening.

Example 2 is the volatile composition releasing assembly of example(s) 1 or any of the preceding or subsequent examples, wherein the airflow is introduced via a suction mechanism that pulls the airflow into the air intake opening.

Example 3 is the volatile composition releasing assembly of example(s) 2 or any of the preceding or subsequent examples, wherein the suction mechanism is created using an internal air-moving device positioned within the housing.

Example 4 is the volatile composition releasing assembly of example(s) 3 or any of the preceding or subsequent examples, wherein the air intake opening is positioned on a suction side of the internal air-moving device.

Example 5 is the volatile composition releasing assembly of example(s) 3 or any of the preceding or subsequent examples, wherein at least one of a size or an operation speed of the internal air-moving device or a size of at least one of the air intake opening or air output opening is adjusted to control at least one of a speed of the portion of the airflow within the housing or a number of cycles that the portion of the airflow moves through the air recirculation pattern.

Example 6 is the volatile composition releasing assembly of example(s) 2 or any of the preceding or subsequent examples, wherein the suction mechanism for the air intake opening is created by the air intake opening being shaped to create a venturi effect that draws the airflow into the housing.

Example 7 is the volatile composition releasing assembly of example(s) 1 or any of the preceding or subsequent examples, wherein the airflow is introduced via an external blowing mechanism that pushes the airflow into the air intake opening.

Example 8 is the volatile composition releasing assembly of example(s) 7 or any of the preceding or subsequent examples, wherein the external blowing mechanism is created using an external air-moving device that directs the airflow into the air intake opening.

Example 9 is the volatile composition releasing assembly of example(s) 8 or any of the preceding or subsequent examples, wherein the external air-moving device comprises at least one of a car vent, HVAC system, free-standing fan, or other air moving devices.

Example 10 is the volatile composition releasing assembly of example(s) 8 or any of the preceding or subsequent examples, wherein the external air-moving device is connected to the housing.

Example 11 is the volatile composition releasing assembly of example(s) 1 or any of the preceding or subsequent examples, wherein the housing further comprises: a first chamber comprising a first air intake opening, a first air output opening, and a first receptacle shaped to receive a first material comprising a volatile composition, the first chamber being shaped to create a first air recirculation pattern within an interior of the first chamber; and a second chamber comprising a second air intake opening, a second air output opening, and a second receptacle shaped to receive a second material comprising a volatile composition, the second chamber being shaped to create a second air recirculation pattern within an interior of the second chamber; wherein the airflow enters the housing through the first air intake opening, and at least a first portion of the airflow follows the first air recirculation pattern, wherein the first portion of the airflow passes over an exposed surface of the first material comprising a volatile composition at least two times before exiting the first chamber through the first air output opening; and wherein the first portion of the airflow enters the second air intake opening, and at least a second portion of the first portion of the airflow follows the second air recirculation pattern, wherein the second portion passes over an exposed surface of the second material at least two times before exiting the second chamber and the housing through the second air output opening.

Example 12 is the volatile composition releasing assembly of example(s) 1 or any of the preceding or subsequent examples, wherein the housing further comprises: a first chamber comprising a first air intake opening, a first air output opening, and a first receptacle shaped to receive a first material comprising a volatile composition, the first chamber being shaped to create a first air recirculation pattern within an interior of the first chamber; and a second chamber comprising a second air intake opening, a second air output opening, and a second receptacle shaped to receive a second material comprising a volatile composition, the second chamber being shaped to create a second air recirculation pattern within an interior of the second chamber; wherein the airflow enters the housing through the first air intake opening and the second air intake opening; wherein at least a first portion of the airflow entering the first air intake opening follows the first air recirculation pattern, wherein the first portion of the airflow passes over an exposed surface of the first material at least two times before exiting the first chamber and the housing through the first air output opening; and wherein at least a second portion of the airflow entering the second air intake opening follows the second air recirculation pattern, wherein the second portion of the airflow passes over an exposed surface of the second material at least two times before exiting the second chamber and the housing through the second air output opening.

Example 13 is the volatile composition releasing assembly of example(s) 1 or any of the preceding or subsequent examples, further comprising at least one energy source, wherein the energy source controls a temperature of the airflow within the housing.

Example 14 is an air purification assembly (which may incorporate features of any of the preceding or subsequent examples) comprising a housing comprising: an air purifying component arranged within an interior of the housing; an air intake opening; and an air output opening; the housing being shaped to create an air recirculation pattern within the interior of the housing; and wherein an airflow enters the housing through the air intake opening, and at least a portion of the airflow follows the air recirculation pattern; and wherein the portion of the airflow following the air recirculation pattern passes through or by the air purifying component at least two times before exiting the housing through the air output opening.

Example 15 is the air purification assembly of example(s) 14 or any of the preceding or subsequent examples, wherein the airflow is introduced via a suction mechanism that pulls the airflow into the air intake opening.

Example 16 is the air purification assembly of example(s) 15 or any of the preceding or subsequent examples, wherein the suction mechanism for the air intake opening is created by the air intake opening being shaped to create a venturi effect that draws the airflow into the housing.

Example 17 is the air purification assembly of example(s) 15 or any of the preceding or subsequent examples, wherein the suction mechanism is created using an internal air-moving device positioned within the housing, and the air intake opening is positioned on a suction side of the internal air-moving device.

Example 18 is the air purification assembly of example(s) 16 or any of the preceding or subsequent examples, wherein at least one of a size or an operation speed of the internal air-moving device may be adjusted to control at least one of a temperature of the portion of the airflow or a number of cycles that the portion of the airflow moves through the air recirculation pattern.

Example 19 is the air purification assembly of example(s) 14 or any of the preceding or subsequent examples, wherein the airflow is introduced via a blowing mechanism that pushes the airflow into the air intake opening, and the blowing mechanism is created using an external air-moving device that directs the airflow into the air intake opening.

Example 20 is the air purification assembly of example(s) 19 or any of the preceding examples, wherein the external air-moving device comprises at least one of a car vent, HVAC system, free-standing fan, or other air moving devices, and wherein the external air-moving device is connected to the housing.

Different arrangements of the components depicted in the drawings or described above, as well as components and steps not shown or described are possible. Similarly, some features and sub-combinations are useful and may be employed without reference to other features and sub-combinations. Embodiments of the invention have been described for illustrative and not restrictive purposes, and alternative embodiments will become apparent to readers of this patent. Accordingly, the present invention is not limited to the embodiments described above or depicted in the drawings, and various embodiments and modifications may be made without departing from the scope of the claims below.

That which is claimed is:

1. A volatile composition releasing assembly comprising:
a material comprising a volatile composition; and
a housing comprising:
a receptacle shaped to receive the material comprising the volatile composition;
an air intake opening; and
an air output opening;
the housing being shaped to create an air recirculation pattern within an interior of the housing; and
wherein an airflow enters the housing through the air intake opening, and at least a portion of the airflow follows the air recirculation pattern; and
wherein the portion of the airflow following the air recirculation pattern passes over an exposed surface of the material comprising the volatile composition at least two times before exiting the housing through the air output opening.

2. The volatile composition releasing assembly of claim 1, wherein the airflow is introduced via a suction mechanism that pulls the airflow into the air intake opening.

3. The volatile composition releasing assembly of claim 2, wherein the suction mechanism is created using an internal air-moving device positioned within the housing.

4. The volatile composition releasing assembly of claim 3, wherein the air intake opening is positioned on a suction side of the internal air-moving device.

5. The volatile composition releasing assembly of claim 3, wherein at least one of a size or an operation speed of the internal air-moving device or a size of at least one of the air intake opening or air output opening is adjusted to control at least one of a speed of the portion of the airflow within the housing or a number of cycles that the portion of the airflow moves through the air recirculation pattern.

6. The volatile composition releasing assembly of claim 2, wherein the suction mechanism for the air intake opening is created by the air intake opening being shaped to create a venturi effect that draws the airflow into the housing.

7. The volatile composition releasing assembly of claim 1, wherein the airflow is introduced via an external blowing mechanism that pushes the airflow into the air intake opening.

8. The volatile composition releasing assembly of claim 7, wherein the external blowing mechanism is created using an external air-moving device that directs the airflow into the air intake opening.

9. The volatile composition releasing assembly of claim 8, wherein the external air-moving device comprises at least one of a car vent, HVAC system, free-standing fan, or other air moving devices.

10. The volatile composition releasing assembly of claim 8, wherein the external air-moving device is connected to the housing.

11. The volatile composition releasing assembly of claim 1, wherein the housing further comprises:
a first chamber comprising a first air intake opening, a first air output opening, and a first receptacle shaped to receive a first material comprising a volatile composition, the first chamber being shaped to create a first air recirculation pattern within an interior of the first chamber; and
a second chamber comprising a second air intake opening, a second air output opening, and a second receptacle shaped to receive a second material comprising a volatile composition, the second chamber being shaped to create a second air recirculation pattern within an interior of the second chamber;
wherein the airflow enters the housing through the first air intake opening, and at least a first portion of the airflow follows the first air recirculation pattern, wherein the first portion of the airflow passes over an exposed surface of the first material comprising a volatile composition at least two times before exiting the first chamber through the first air output opening; and
wherein the first portion of the airflow enters the second air intake opening, and at least a second portion of the first portion of the airflow follows the second air recirculation pattern, wherein the second portion passes over an exposed surface of the second material at least two times before exiting the second chamber and the housing through the second air output opening.

12. The volatile composition releasing assembly of claim 1, wherein the housing further comprises:
a first chamber comprising a first air intake opening, a first air output opening, and a first receptacle shaped to receive a first material comprising a volatile composition, the first chamber being shaped to create a first air recirculation pattern within an interior of the first chamber; and
a second chamber comprising a second air intake opening, a second air output opening, and a second receptacle shaped to receive a second material comprising a volatile composition, the second chamber being shaped to create a second air recirculation pattern within an interior of the second chamber;
wherein the airflow enters the housing through the first air intake opening and the second air intake opening;
wherein at least a first portion of the airflow entering the first air intake opening follows the first air recirculation pattern, wherein the first portion of the airflow passes over an exposed surface of the first material at least two times before exiting the first chamber and the housing through the first air output opening; and
wherein at least a second portion of the airflow entering the second air intake opening follows the second air recirculation pattern, wherein the second portion of the airflow passes over an exposed surface of the second material at least two times before exiting the second chamber and the housing through the second air output opening.

13. The volatile composition releasing assembly of claim 1, further comprising at least one energy source, wherein the energy source controls a temperature of the airflow within the housing.

14. An air purification assembly comprising a housing comprising:
an air purifying component arranged within an interior of the housing;
an air intake opening; and
an air output opening;
the housing being shaped to create an air recirculation pattern within the interior of the housing; and
wherein an airflow enters the housing through the air intake opening, and at least a portion of the airflow follows the air recirculation pattern; and
wherein the portion of the airflow following the air recirculation pattern passes through or by the air purifying component at least two times before exiting the housing through the air output opening.

15. The air purification assembly of claim 14, wherein the airflow is introduced via a suction mechanism that pulls the airflow into the air intake opening.

16. The air purification assembly of claim 15, wherein the suction mechanism for the air intake opening is created by the air intake opening being shaped to create a venturi effect that draws the airflow into the housing.

17. The air purification assembly of claim 15, wherein the suction mechanism is created using an internal air-moving device positioned within the housing, and the air intake opening is positioned on a suction side of the internal air-moving device.

18. The air purification assembly of claim 17, wherein at least one of a size or an operation speed of the internal air-moving device may be adjusted to control at least one of a temperature of the portion of the airflow or a number of cycles that the portion of the airflow moves through the air recirculation pattern.

19. The air purification assembly of claim 14, wherein the airflow is introduced via a blowing mechanism that pushes the airflow into the air intake opening, and the blowing mechanism is created using an external air-moving device that directs the airflow into the air intake opening.

20. The air purification assembly of claim 19, wherein the external air-moving device comprises at least one of a car vent, HVAC system, free-standing fan, or other air moving devices, and wherein the external air-moving device is connected to the housing.
